(19) United States
(12) Patent Application Publication
(10) Pub. No.: US 2014/0155349 A1
(43) Pub. Date: Jun. 5, 2014

(54) Health Care

(71) Applicants: Anthony I. Rozmanith, Winchester, MA (US); Jolan S. Rozmanith, Winchester, MA (US)

(72) Inventors: Anthony I. Rozmanith, Winchester, MA (US); Jolan S. Rozmanith, Winchester, MA (US)

(21) Appl. No.: 13/913,891

(22) Filed: Jun. 10, 2013

Related U.S. Application Data
(63) Continuation of application No. 12/671,072, filed on Jan. 28, 2010, now abandoned.

(51) Int. Cl. A61K 31/60; A61K 31/702; A61K 31/155; A61K 31/192; A61K 31/18

(52) U.S. Cl. CPC A61K 31/60; A61K 31/192; A61K 31/18; A61K 31/155; A61K 31/702
USPC 514/61; 514/163; 514/162

(57) ABSTRACT

The use of a pharmaceutical regimen to deter in an individual, as the individual ages, the development of a cardiovascular disease (CVD) or Alzheimer's disease or to treat an individual who has CVD or Alzheimer's, the individual ingesting on a daily basis a regimen comprising pharmaceutically effective amounts of magnesium salicylate and naproxen, the amount of the magnesium salicylate being no greater than about 260 mg, and also the use of a pharmaceutical regimen to treat an individual who has Type 2 diabetes, the regimen being ingested daily by the individual and comprising pharmaceutically effective amounts of at least one glucose-lowering drug and magnesium salicylate in an amount no greater than about 260 mg.

### CROSS REFERENCE TO RELATED APPLICATION

This application is a continuation of application Ser. No. 12/671,072, filed Jan. 28, 2010, which is the national stage of International Patent Application No. PCT/US09/55984, filed Sep. 4, 2009, which claims the benefit of U.S. Provisional Patent Application No. 61/094,290, filed Sep. 4, 2008.

### FIELD OF THE INVENTION

The present invention relates to the field of health care. More particularly, it relates to the use of pharmaceutical regimens for deterring the development in individuals of cardiovascular disease or of Alzheimer's disease and for treating individuals who are afflicted with such diseases and also for treating individuals who suffer from Type 2 diabetes.

It has been reported that more than 70 million individuals in the United States alone have one or more forms of cardiovascular disease. The most common form of heart disease is reported to be coronary heart disease (CHD) which is caused by a narrowing of the coronary arteries that carry blood to the heart. It has been reported that about 7 million Americans suffer from CHD which is considered to be the cause of the deaths of more than 500,000 men and women in the United States.

Alzheimer's disease is the most common form of dementia. It has been reported that about 5 million individuals in the United States suffer from the effects of Alzheimer's.

Another devastating disease is Type 2 diabetes which, according to reports, afflicts more than 350 million individuals worldwide. The number of individuals who reside in the U.S. and who are reported to suffer from Type 2 diabetes is reported to be over 20 million.

The present invention relates to the treatment of individuals who suffer from CVD, Alzheimer's disease or Type 2 diabetes and to deterring the development of CVD and Alzheimer's disease in individuals.

### SUMMARY OF THE INVENTION

The present invention provides a method for:

(A) deterring in an individual, as the individual ages, the development of a bodily condition which is a pre-cardiovascular indicator of the development of a cardiovascular disease (CVD); or

(B) treating an individual who exhibits a bodily condition which is a pre-cardiovascular indicator of the development of CVD or an individual who has a bodily condition which is possessed by an individual who has CVD; wherein said individual:
(i) ingests one or more drugs which are effective to deter the development of said condition or to reduce the severity of an existing condition; and
(ii) optionally lives a life style which deters also the development of said condition or reduces the severity of an existing condition, the improvement comprising:
   the practice by the individual of a regimen which comprises the ingestion on a daily basis by the individual of pharmaceutically effective amounts of magnesium salicylate and naproxen, the amount of the magnesium salicylate being no greater than about 260 mg. In preferred form, the daily regimen includes the use of about 80 to about 260 mg of magnesium salicylate and about 160 to about 600 mg of naproxen.

Another aspect of the present invention is the use of the aforementioned regimen to deter in an individual, as the individual ages, the development of Alzheimer's disease or to treat an individual who has the symptoms of Alzheimer's disease.

Still another aspect of the present invention is the provision of a method for treating an individual who has Type 2 diabetes comprising the practice by the individual of a regimen which includes the ingestion daily by the individual of:

(A) at least one glucose-lowering drug (G-LD) which functions to lower the amount of glucose in the blood of the individual; and

(B) a non-steroidal anti-inflammatory drug (NSAID), namely magnesium salicylate; and optionally an additional NSAID;

(C) wherein on a daily basis, the G-LD(s) is ingested in an amount of about 1 to about 3000 mg and the magnesium salicylate is ingested in an amount of about 80 to about 260 mg, the total amount ingested of the G-LD(s) and the NSAID(s) being a pharmaceutically effective amount; and

wherein said individual optionally lives a life style which reduces the severity of undesirable symptoms associated with Type 2 diabetes. Examples of G-LDs are a sulfonylurea, metformin, and acarbose.

Another aspect of the present invention is the provision of a regimen which is useful for treating an individual to reduce the risk of the development of a cardiovascular disease or the development of Alzheimer's disease or to treat an individual who has a cardiovascular disease or Alzheimer's disease, the regimen being in a form for oral ingestion by an individual and comprising:

(A) about 80 to about 260 mg of a non-steroidal anti-inflammatory drug (NSAID), namely magnesium salicylate;

(B) about 160 to about 600 mg of another non-steroidal anti-inflammatory drug, namely naproxen; and optionally

(C) one or more additional NSAIDs in an amount no greater than about 20 wt. % of the total amount of the NSAIDs comprising the regimen.

An additional embodiment of the present invention is the provision of a regimen which is useful for treating an individual who has Type 2 diabetes, the regimen being in a form for oral ingestion by an individual and comprising:

(A) about 1 to about 3000 mg of at least one glucose-lowering drug which functions to lower the amount of glucose in the blood of the individual;

(B) about 80 to about 260 mg of a non-steroidal anti-inflammatory drug (NSAID), namely magnesium salicylate; and optionally

(C) one or more additional NSAID(s) in an amount no greater than about 20 wt. % of the total amount of the NSAIDs comprising the regimen.

Still additional embodiments of the present invention comprise the provision of the following compositions;

(I) A composition for use by an individual to deter the onset of CVD and/or Alzheimer's disease or for use by an individual who has CVD and/or Alzheimer's disease and comprising:

(A) about 12 to about 62 wt. % of the NSAID Mg salicylate;

(B) about 38 to about 88 wt. % of the NSAID naproxen; and optionally

(C) one or more additional NSAID(s) in an amount that comprises no more than about 20 wt. % of the total amount of NSAIDs comprising the composition; and

(II) A composition for use in treating a Type 2 diabetic comprising:

(A) about 0.4 to about 97 wt. % of at least one glucose-lowering drug;

(B) about 2.6 to about 99 wt. % of the NSAID Mg salicylate; and optionally

(C) one or more additional NSAIDs in an amount that comprises no more than about 20 wt. % of the total amount of NSAIDs comprising the composition.

### DETAILED DESCRIPTION OF THE INVENTION

As described in detail below, the present invention can be used to treat an individual to reduce the risk of the development of a cardiovascular disease or the development of Alzheimer's disease or to treat an individual that has a cardiovascular disease or Alzheimer's disease. In addition, the present invention can be used to treat an individual who has Type 2 diabetes.

The term “a cardiovascular disease” (also referred to herein as “CVD” for convenience) is used in the broad sense to include a disease which affects adversely the heart or a blood vessel, that is, heart disease (known also as “cardiac disease”) or a disease of one or more blood vessels (known also as “vascular disease”).

Examples of heart disease include: angina; arrhythmia; congenital heart disease; coronary artery disease (CAD); dilated cardiomyopathy; heart attack (myocardial infarction); heart failure; hypertrophic cardiomyopathy; mitral regurgitation; mitral valve prolapse; and pulmonary stenosis.

Examples of vascular disease include carotid artery disease (stroke precursor), peripheral artery disease, and any other condition that results in the blockage of fresh (that is, oxygenated) blood supply to the heart or brain.

With regard to CVD, various types of individuals can benefit by the use of the present invention. For example, the present invention can be used to treat any individual who is afflicted with one or more bodily conditions which are pre-cardiovascular indicators of the development of CVD. Such bodily conditions are known. Examples of such bodily conditions are high blood pressure, obesity, and Type 2 diabetes. An individual who has a pre-disposition to the development of CVD as a result of genetic makeup can benefit also by use of the present invention. Also, it is well established that an individual can engage in conduct which causes the development of a bodily condition which is not readily apparent or identifiable, but which exists, nevertheless, and leads to the development of CVD, including death without warning. Various types of such conduct are known in the art; examples are smoking, insufficient or inadequate daily exercise, and becoming obese. In addition, an individual may intentionally or even unintentionally be exposed to disease-causing environmental conditions, for example, being exposed to radon gas or to aromatic or halogenated solvents.

An individual who does not have CVD or a bodily condition which is a pre-cardiovascular indicator of the development of CVD can benefit also by use of the present invention in a prophylactic way, that is, to deter the development of a bodily condition which is associated with the cause of CVD. An example of such an individual is one who appears to be healthy in the cardiovascular sense and who lives a life style which is recognized as being effective in reducing the risk of the development of one or more bodily conditions which lead to CVD. The use of the present invention by such an individual can be effective in delaying the onset or warding off the development of such bodily condition that tends to develop naturally as an individual ages. It is recommended that the present invention be used with regularity in a prophylactic way by a “healthy” individual who reaches the age of about 30 and thereafter.

In addition, the present invention can be used to treat effectively an individual who is not taking medication of the type which is effective in treating a bodily condition which is associated with CVD, for example, medication which controls the level of cholesterol and/or which is a blood thinner (anti-coagulant) and/or which is associated with kidney or liver damage. Nevertheless, the present invention can be used also in combination with such medications, depending on how the individual responds to treatment with only the regimen of the present invention.

In addition, the present invention can be used to treat any individual who has CVD. Such use can mitigate the adverse effects of CVD and/or deter the worsening of the condition or conditions associated with CVD.

As regards Alzheimer's disease (also referred to herein as “Alzheimer's”), it is accepted that this disease is the most common cause of the loss of mental function in individuals of age 65 and over. The disease is a brain disorder in which the individual suffers, for example, from loss of memory, language skills, and perception of time and space; such losses worsen with time. Eventually the individual is unable to care for him or herself. Although the disease exists in the main in individuals aged 65 and older, earlier-onset Alzheimer's is known also to exist in younger individuals, although rarely. As individuals grow older, the risk of developing the disease increases.

There are various explanations as to why an individual becomes afflicted with Alzheimer's. For example, it has been established that there are particular groups of individuals who are more susceptible to developing the disease (high-risk individuals) than individuals who comprise the general population. For example, research has shown that individuals who have a family history of Alzheimer's are more apt to develop the disease than individuals who do not have such family history. Another exemplary group of such high-risk individuals consists of those who have suffered brain injuries, for example, from vehicle crashes, falls, impacts to the head, and hemorrhages from burst blood vessels. Research has shown that individuals who have suffered such brain trauma often develop in later life dementia, including, for example, Alzheimer's. Still another group of such high-risk individuals are Type 2 diabetics; it has been reported that they have two to four times the risk for developing Alzheimer's and that, in incipient Alzheimer's, conditions are accelerated in Type 2 diabetics relative to the general population.

There has been a very substantial amount of research conducted on the brains of individuals who have been diagnosed with Alzheimer's and who have died. Such research has revealed, among other things, that “such” brains have characteristics which are distinctive. For example, they include proteins which are abnormally shaped, including, for example, abnormally shaped proteins which are called “plaques” (also referred to as “senior plaque deposits”) and which are formed mostly in the regions of the brain that are related to the function of memory. It has been reported that an amyloid precursor protein forms toxic plaques which cause neurons in the brains of individuals with Alzheimer's to shrink and eventually die; it is believed that this leads to some of the terribly undesirable symptoms of the disease.

An important benefit associated with the use of the present invention in connection with its applicability to CVD is that it can be effective also in postponing the development in an individual of Alzheimer's or prevent its development; this is an example of the prophylactic use of the present invention. In addition, the present invention can be used to advantage to treat an individual who is afflicted with Alzheimer's. It is believed that this is due, at least in part, to the effectiveness of the regimen of the present invention to control in the individual, particularly Type 2 diabetics, the level of blood sugar (glucose) and/or to slow the formation in the brain of senior plaque deposits.

The regimen of the present invention includes the use of magnesium (Mg) salicylate (the NSAID), preferably in its tetrahydrate form which is available commercially as efflorescent colorless crystals; they are soluble in water and alcohol. Mg salicylate is well known and is available in the U.S. over the counter for use in relieving pain and inflammation attributed to various conditions, for example, arthritis, bursitis, and tendenitis.

For use in the regimen of the present invention, the Mg salicylate can be administered orally, for example, as a tablet, gelcap, or caplet, or it can be administered as a component of a composition which contains one or more other ingredients of the regimen and which is in any suitable form associated with pharmaceutical compositions that are taken orally.

The daily amount of Mg salicylate used in the regimen of the present invention is a pharmaceutically effective amount, but an amount not greater than about 260 mg, for example, an amount within the range of about 80 to about 260 mg on a daily basis. The Mg salicylate can be ingested once daily or more than once, for example, twice a day, and preferably at breakfast or with another meal. As explained hereinafter, the particular amount used will depend on various factors.

For use in treating CVD or Alzheimer's, the regimen of the present invention also includes the use of another NSAID, namely naproxen, which is a compound that is compatible with Mg salicylate. Naproxen comprises the molecule C₁₂H₁₄O₃; it is available also as a sodium salt. Naproxen is a popularly used drug that is effective in alleviating pain, reducing fever, and inflammation and stiffness caused by many types of conditions; for example, various types of arthritis and bursitis. Naproxen functions by reducing hormones that cause bodily inflammation and pain. It is available in immediate-release and delayed-release form. One form of naproxen is sold under the trademark NAPROSYN®.

For use in the regimen of the present invention, naproxen can be administered orally, for example, as a tablet, gelcap, or caplet, or it can be administered as a component of a composition which contains one or more other ingredients of the regimen and which is in any suitable form associated with pharmaceutical compositions that are taken orally.

The daily amount of naproxen used in the regimen of the present invention is a pharmaceutically effective amount, but an amount not greater than about 600 mg, for example, an amount within the range of about 160 to about 600 mg on a daily basis. The naproxen can be ingested once daily or more than once, for example, twice a day. The particular amount of naproxen used will depend on various factors, as explained hereinafter.

For use in treating CVD or Alzheimer's, it is preferred that Mg salicylate and naproxen comprise about 100% of the NSAIDs used. However, the regimen can include optionally one or more additional NSAIDs Examples of additional NSAIDs are set forth below.

| Generic Name | TRADENAME |
|---|---|
| diclofenac | Cataflam, Voltaren, Arthrotec (combined with |
| | misoprostol) |
| diflunisal | Dolobid |
| etodoiac | Lodine, Lodine XL |
| fenoprofen | Nalfon, Nalfon 200 |
| flurbiprofen | Ansaid |
| ibuprofen | Motrin, Tab-Profen, Vicoprofen (combined with |
| | hydrocodone), Combunox (combined with oxycodone) |
| indomethacin | Indocin, Indocin SR, Indo-Lemmon, Indomethagan |
| ketoprofen | Oruvall |
| ketorolac | Toradol |
| mefenamic | Ponstel |
| acid | |
| meloxicam | Mobic |
| nabumetone | Relafen |
| oxaprozin | Daypro |
| piroxicam | Feldene |
| sulindac | Clinoril |
| tolmetin | Tolectin, Tolectin DS, Tolectin 600 |

If used, the additional NASID(s) should be present in the regimen in an amount of no more than about 20 wt. % of the total amount of the NSAIDs used in the regimen.

A regimen of the present invention comprising Mg salicylate, a glucose-lowering drug (G-LD), and, optionally naproxen, can be used to treat an individual'who has Type 2 diabetes, that is, a disease that is characterized by elevated blood glucose levels, for example, levels of HbA1c of 7 (or higher if untreated), as measured by glucose bound hemoglobin. (The term “HbA1c” refers to a test that measures the average amount of glycated hemoglobin in the blood over about a 3-month period; it is formed when glucose attaches to hemoglobin.) Examples of symptoms in individuals who have Type 2 diabetes include blurred vision or other eye diseases, including the development of blindness, increased thirst and urination, weight loss, aches in feet or hands and kidney problems. In addition, it has been observed that individuals with Type 2 diabetes have a 3 to 5 times greater propensity for developing CVD and, as mentioned above, 2 to 4 times the risk of developing Alzheimer's, than those who do not have this disease.

For treating Type 2 diabetes, there can be used a regimen comprising Mg salicylate and one or more of a G-LD, including, for example, those which function in different ways; such drugs are well known. Examples of glucose-lowering drugs that can be used in the practice of the present invention include metformin, acarbose, and a sulfonylurea. A mixture of G-LD(s) can be used also, for example, metformin and acarbose, with or without a sulfonylurea. A preferred regimen includes the use of both metformin and a sulfonylurea.

The appropriate use of a glucose-lowering drug in the regimen of the present invention is effective in lowering elevated levels of glucose in the blood to near normal ranges; such control of the glucose levels has the effect of improving the quality of life of a Type 2 diabetic and giving the individual the opportunity to live a relatively long and healthy life. This is accomplished by eliminating or lessening the adverse symptoms associated with Type 2 diabetes. The use of the regimen of the present invention should be accompanied preferably by the adoption of the Type 2 diabetic of a lifestyle that is medically recommended for Type 2 diabetics, for example, eating a balanced diet that limits and spreads carbohydrate ingestion throughout the day, engaging in regular exercise, refraining from smoking, and limiting caloric intake.

The daily amount of the glucose-lowering drug(s) used in the regimen of the present invention is a pharmaceutically effective amount, that is, an amount that will reduce the blood glucose level to a normal or near normal range. The particular amount of the glucose-lowering drug used will depend on the drug used and, as explained hereinafter, on various other factors. By way of example, it is recommended that a sulfonylurea, for example, glyburide be used in an amount within the range of about 1 to about 20 mg, preferably an amount within the range of about 1 to about 6 mg. Metformin is generally recommended to be used daily in an amount within the range of about 500 to about 2,550 mg. Acarbose is generally recommended to be used daily in an amount in the range of about 50 to about 300 mg. Taking into account that there are other available glucose-lowering drugs, it is believed that the most widely used regimens will include an amount of a glucose-lowering drug or a mixture of such drugs that falls within the range of about 1 to about 3,000 mg on a daily basis.

The glucose-lowering drug can be used conveniently and effectively by oral ingestion of, for example, a tablet; accordingly, the injection into the blood stream of insulin or the like is not necessary, that is, the use of the regimen is “injection-free” in its preferred form. In special cases, injection of the drug may be necessary, such as, for example, in severe cases of the disease which require hospitalization or during pregnancy or breast feeding. The glucose-lowering drug can be administered also as a component of a composition which contains one or more other constituents of the regimen and which is in any suitable form associated with pharmaceutical compositions.

The regimen of the present invention can include also the use of other constituents that are considered healthy for inclusion in the diet of an individual, for example, vitamins, minerals, and supplements, including, for example, dark chocolate, fish oil, garlic, moderate amounts of red wine, and red grapes, including their components or extracts.

In addition, a particular individual may have a health condition or disease which is being treated with one or more drugs pursuant to medical advice. The regimen of the present invention can include also the use of a prescribed medication which is compatible with other drugs of the regimen, including the NSAID(s) of the regimen. For example, the regimen can include drugs which are designed to treat hypertension, for example, enalapril maleate (an ACE inhibitor), toprol/metoprolol tartrate and other beta blockers. An individual who has a condition involving benign prostrate enlargement (BHP) can include in the regimen, for example, FLOWMAX, HYTRIN, or CARDURA (doxazosin mesylate, an alpha-1-blocker); the last mentioned drug has dual functions in that it increases urinary flow and reduces high blood pressure. The use of the regimen of the invention can be accompanied also by treatment of glaucoma with one or more appropriate drugs, for example, an eye-drop solution of timolol, brimonidine tartrate, or xalatan. Two or more of “glaucoma” drugs can be used. It should be understood that the aforementioned are exemplary and that other medications can be used with the present regimen as may be desirable or necessary for the treatment of an ailment in the individual.

The amount of such other constituent to include in the regimen or that accompanies the use of the regimen is a pharmaceutically effective amount, as determined by those skilled in the art.

As mentioned above, the particular amount of drug used in the regimen depends on a number of factors; consider the following as general guidelines. For example, when using the regimen in a prophylactic way, the drugs can be used in amounts toward the lower end of the amount range, particularly in early stage Type 2 diabetes. In situations in which the individual has one or more bodily conditions or engages in conduct that are pre-cardiovascular indicators of the development of CVD, the drugs can be used in amounts within the middle of the amount range. Similarly, with individuals who are considered at high risk to develop Alzheimer's, the drugs can be used in amounts within the middle of the amount range. For those who have already developed CVD or Type 2 diabetes or Alzheimer's, the drugs can be used in amounts toward the higher end of the amount range. Similarly, the drugs can be used in amounts toward the higher end of the amount range with older individuals who are afflicted with CVD or Type 2 diabetes or Alzheimer's. Typically, individuals who have a relatively high body mass index (BMI), that is, a BMI which characterizes the individual as overweight or obese, will also benefit by use of the present invention. As mentioned above, the aforementioned statements respecting “amounts of drugs” should be considered general guidelines. For any particular individual, adjustments in the amounts of drugs used can be made as test results show that adjustment is warranted.

The regimen of the present invention can be in any suitable form that is used typically in medical applications. For example, each constituent comprising the regimen can be contained in an individual package, including, for example, a package which is associated with one or more packages containing other of the constituents comprising the regimen, for example, a plurality of boxed packages. Also, the regimen can be in the form of a composition which comprises two or more of the constituents of the regimen and which is in the form of a typical pharmaceutical composition, for example, a tablet, gel cap, pill, etc.

A composition for use which involves the CVD and/or the Alzheimer's aspects of the present invention can comprise:

(A) about 12 to about 62 wt. % of Mg salicylate; and

(B) about 38 to about 88 wt. % of naproxen; and optionally

(C) other ingredients of the type exemplified above.

In a composition which includes one or more additional NASIDs, the additional NSAID(s) should comprise no more than about 20 wt. % of the total amount of NSAIDs comprising the composition.

A composition for use in treating a Type 2 diabetic according to the present invention can comprise:

(A) about 0.4 to about 97 wt. % of a glucose-lowering drug; and

(B) about 2.6 to about 99 wt. % of Mg salicylate; and optionally

(C) one or more other ingredients of the type exemplified above.

In a composition which includes one or more additional NASIDs, the additional NSAID(s) should comprise no more than about 20 wt. % of the total amount of NSAIDs comprising the composition.

### EXAMPLES

The following examples are illustrative of the practice of the present invention.

There are set forth in the Table below the results of tests of blood and urine collectable from a male individual (test subject) who was born in 1927 and who was diagnosed with Type 2 diabetes in 1984. The individual has a family history of cardiovascular disease (CVD); the death of each parent was caused by a stroke (mother at age 62 and father at age 86). The individual has not suffered any affliction associated with CVD during his entire lifetime; he has never used statins. The body weight of the individual at age 80 ranged from 192 to 198 lbs., with a BMI of 28-33 at 1.70 m height.

The test results reported in the Table include test results of “Initial Tests” and three “Additional Tests” which are carried out 176, 352, and 882 days after the Initial Tests, as indicated in the Table.

The daily regimen of the present invention associated with the test results which are reported in the Table is described below.

The regimen includes magnesium salicylate tetrahydrate which is taken orally in the form of one 150 mg tablet; the tablet is taken usually at breakfast or, on occasions, in split dosages with two or more meals. The regimen includes also naproxen (NAPROSYN®). Optionally, a third NSAID is taken for added pain relief, for example, celecoxib (CELEBREX) in a 200 mg capsule over time as needed.

In addition, the daily regimen includes three different drugs which function to control elevated blood sugar levels in individuals, namely: (A) glyburide which is an oral blood-glucose-lowering drug of the sulfonyl urea class; (B) metformin which is a compound that belongs to a group of diabetes medicines called biguanides; and (C) acarbose which is an alphaglucosidase inhibitor that functions to lower glucose in individuals. The glyburide (MICRONASE®) is taken orally in the form of a 2.5 mg tablet once daily at breakfast, including a substantial amount of liquid, for example, a cup or more of tea. The metformin is also taken orally with meals, including liquids, in the form of an 850 mg tablet (Glucophage HCl brand) that is taken 3 times a day. The acarbose (PRECOSE®) is also taken orally in the form of a 100 mg tablet that is split and that is taken twice daily at breakfast and dinner.

In addition to the aforementioned “anti-CVD/Alzheimer's” and “glucose-lowering” drugs, the regimen includes also other drugs that function to treat other bodily conditions; this exemplifies that the present invention can be used in a compatible way with other drugs. There follows a description of such other drugs.

(A) oral ingestion of doxazosin mesylate in the form of either a 2 mg or 8 mg tablet (CARDURA) which can be taken once daily in the morning (increases urinary flow in individuals with benign hyperprostatitise enlargement (BHP). CARDURA is also a hypertension drug);

(B) oral ingestion of enalapril maleate in the form of a 20 mg tablet which can be taken twice daily (hypertension drug);

(C) oral ingestion of metoprolol tartrate, a beta-blocker in the form of a 100 mg tablet which can be taken three times daily (hypertension drug); and

(D) the following drugs in the form of eye-drop solutions which can be applied as indicated:
(1) timolol (TIMOLOL GFS) 0.5%—one drop, each eye, in the morning and in the evening to treat glaucoma;
(2) brimonidine tartrate (ALPHAGAN)—one drop, each eye, 3 times daily to treat glaucoma; and
(3) xalatan (LATANOPROST)—one drop, each eye, at bed time to treat glaucoma.
   During the period of use of the regimen, there can be a departure from the afore-described regimen in that the amount of doxazosin mesylate (used to increase urinary flow) can be changed from 2 mg/day to 8 mg/day and an additional departure can be the replacement of the use of doxazosin with tamsulosin hydrochloride (FLOMAX) in a daily dose of 8 mg/day taken after breakfast.

Comments follow on various of the tests which are the subject of the Table below. Test A, that is, HbA1c, is a test which, as mentioned above, is a test that measures the amount of glycated hemoglobin in the blood. This is a significant test in that it indicates the average level of an individual's glucose over a relatively long period of time, for example, over a several month period. The BUN test refers to the “blood urea nitrogen” test which is used to evaluate how well an individual's kidneys are functioning; it measures the amount of nitrogen in the individual's blood. The CRP cardio test refers to a test that measures vascular inflammation which is a strong indicator of future cardiovascular events for the test subject. The table includes also the results of testing the individual for total cholesterol, triglyercides, and HDL and LDL cholesterol, often referred to respectively as “good” and “bad” cholesterol.

The Table includes also a column entitled “Reference” which identifies ranges of test values which are associated with reduced risk of developing or worsening of conditions of the involved diseases.

TABLE
Additional Tests, Days After Initial Tests

| | | | | | |
|---|---|---|---|---|---|
| Test | Initial Tests | 176 | 352 | 882 | Reference |

Test A (long-term blood sugar)

| | | | | | |
|---|---|---|---|---|---|
| HbA1c | 5.6% | 5.6% | 6.1% | 5.8% | 4.2-5.8% |

Other Tests (random sample)

| BUN | 15 | mg/dL | NA* | | 16 | mg/dL | 12 | mg/dL | 6-20 | mg/dL |
|---|---|---|---|---|---|---|---|---|---|---|
| cholesterol | 139 | mg/dL | 126 | mg/dL | 140 | mg/dL | 137 | mg/dL | <200 | mg/dL |
| triglycerides | 307 | mg/dL | 180 | mg/dL | 224 | mg/dL | 174 | mg/dL | <200 | mg/dL |
| CRP cardio | 0.4 | mg/L | NA | | 0.4 | mg/L | NA | | 0.1-5.0 | mg/L |
| HDL cholesterol | 26 | mg/dL | 22 | mg/dL | 25 | mg/dL | 28 | mg/dL | 35-55 | mg/dL |
| LDL cholesterol | 52 | mg/dL | 68 | mg/dL | 70 | mg/dL | 74 | mg/dL | <130 | mg/dL |

*not available

It is submitted that the test results which are reported in the above Table speak for themselves as they are compared with the “Reference” values. Test values which fall within the range of the “Reference” values are considered in general to be acceptable. With regard to the HbA1 c, a relatively high value, for example, greater than about 8%, means generally that the individual is at risk of developing diabetes complications. With regard to the test results for the CRP cardio test, it has been reported that test values which are relatively high (>3 to 5.0 mg/L) increases the individual's CHD (coronary heart disease) risk.

In addition to the test results reported in the above Table, the test subject was evaluated also in the initial and last series of tests (882 days after Initial Tests) for CHD Risk Assessment Factor. CHD refers to “coronary heart disease” which is reported to be the most common form of heart disease and which is caused by a narrowing of the coronary arteries that feed the heart. As is known, the “CHD” assessment takes into account many aspects of the test subject's physical conditions and life style. The “Reference” value range for a CHD Risk Assessment Factor (average) for a male is considered to be 5.0 to 9.5. The initial test value for the test subject was 5.3 and a value of 4.9 for the last test.

The above Table contains information that provides guidance respecting the use of pharmaceutically effective amounts of the regimen of the present invention. The information is included in the column entitled “Reference” which identifies test values which, as mentioned above, are associated with a reduced risk of developing or worsening of conditions associated with CVD, Alzheimer's, and Type 2 diabetes. As is common in the medical treatment of individuals, adjustments in dosage of the pharmaceutical regimen may be needed, depending on how a patient reacts to a particular prescribed dose. Similarly, in the use of the regimens of the present invention, adjustments in the regimens can be made as the test results for individuals become available. Speaking generally, results for the tests HbA1c and CRP cardio are of particular significance in considering dosage changes.

It should be appreciated that the present invention provides a significant advance in health care and will provide for innumerable individuals the opportunity to live a longer and enjoy a higher quality life.

1. (canceled)

2. A method for:
(A) deterring in an individual, as the individual ages, the development of a bodily condition which is a pre-cardiovascular indicator of the development of a cardiovascular disease (CVD); or
(B) treating an individual who exhibits a bodily condition which is a pre-cardiovascular indicator of the development of CVD or an individual who has a bodily condition which is possessed by an individual who has CVD;
wherein said individual:
(i) ingests one or more drugs which are effective to deter the development of said condition or to reduce the severity of an existing condition; and
(ii) optionally lives a life style which deters also the development of said condition or reduces the severity of an existing condition, the improvement comprising:
the practice by the individual of a regimen which comprises the ingestion by the individual of:
(iii) a non-steroidal anti-inflammatory drug (NSAID), namely magnesium salicylate; and
(iv) another NSAID, namely naproxen;
wherein, on a daily basis, the magnesium salicylate is ingested in an amount of about 80 to about 260 mg and naproxen is ingested in an amount of about 160 to about 600 mg; and wherein, on a daily basis, the total amount ingested of said NSAIDs is a pharmaceutically effective amount.

3. A method according to claim 2 wherein said regimen includes an additional NSAID in an amount of no more than about 20 wt. % of the total amount of the NSAIDs used in the regimen.

4. A method according to claim 2, wherein the magnesium salicylate is in its tetrahydrate form.

5. A method according to claim 2, wherein the individual exhibits a bodily condition which is a pre-cardiovascular indicator of the development of CVD.

6. A method according to claim 2, wherein the individual has a bodily condition which is possessed by an individual who has CVD.

7. A method according to claim 2, wherein the regimen is ingested by an individual who is at least about 30 years old.

8. A method according to claim 2, wherein the individual lives a life style as set forth in (B)(ii) of claim 2.

9. A method for treating an individual who has Type 2 diabetes comprising the practice by the individual of a regimen which includes the ingestion daily by the individual of:
(A) at least one glucose-lowering drug (G-LD) which functions to lower the amount of glucose in the blood of the individual; and
(B) a non-steroidal anti-inflammatory drug (NSAID), namely magnesium salicylate; and optionally an additional NSAID;
(C) wherein on a daily basis, the G-LD is ingested in an amount of about 1 to about 3000 mg and the magnesium salicylate is ingested in an amount of about 80 to about 260 mg; and wherein on a daily basis, the total amount ingested of the G-LD and the NSAID is a pharmaceutically effective amount; and
wherein said individual optionally lives a life style which reduces the severity of undesirable symptoms associated with Type 2 diabetes.

10. A method according to claim 9, wherein the regimen includes naproxen as an additional NSAID in an amount of about 160 to about 600 mg.

11. A method according to claim 9, wherein the individual lives a life style which reduces the severity of said symptoms.

12. A method according to claim 9, wherein the regimen includes a sulfonylurea.

13. A method according to claim 12, wherein the regimen includes about 1 to about 20 mg of glyburide.

14. A method according to claim 13, wherein the regimen includes about 1 to about 6 mg of the glyburide.

15. A method according to claim 9, wherein the regimen includes metformin (G-LD) or acarbose (G-LD) or a mixture thereof.

16. A method according to claim 9 wherein the regimen includes a mixture of a sulfonylurea and metformin.

17.-20. (canceled)

21. A regimen which is useful for treating an individual to reduce the risk of the development of a cardiovascular disease or the development of Alzheimer's disease or to treat an individual who has a cardiovascular disease or Alzheimer's disease, the regimen being in a form for oral ingestion by an individual and comprising:
(A) about 80 to about 260 mg of a non-steroidal anti-inflammatory drug (NSAID), namely magnesium salicylate;
(B) about 160 to about 600 mg of another non-steroidal anti-inflammatory drug, namely naproxen; and optionally
(C) one or more additional NSAIDs in an amount no greater than about 20 wt. % of the total amount of the NSAIDs comprising the regimen.

22.-25. (canceled)
